(19) United States
(12) Patent Application Publication
(10) Pub. No.: US 2024/0341801 A1
(43) Pub. Date: Oct. 17, 2024

(54) FASCIOTOMY KNIFE AND RETRACTOR AND METHOD OF USING SAME

(71) Applicant: Frey Innovations LLC, Haddonfield, NJ (US)

(72) Inventors: Steven Frey, Haddonfield, NJ (US); Alexander Evan Frey, Haddonfield, NJ (US)

(73) Assignee: Frey Innovations LLC, Haddonfield, NJ (US)

(21) Appl. No.: 18/634,103

(22) Filed: Apr. 12, 2024

Related U.S. Application Data
(60) Provisional application No. 63/458,959, filed on Apr. 13, 2023.

(51) Int. Cl. A61B 17/3211; A61B 17/00; A61B 17/02; A61B 17/3209; A61B 90/00

(52) U.S. Cl. CPC A61B 17/3211; A61B 17/0206; A61B 17/32093; A61B 90/361; A61B 2017/00831; A61B 2017/32113

(57) ABSTRACT

A fasciotomy knife is provided and includes an elongated shaft, a holding tab portion at one end of the shaft, and a knife head at a second end of the shaft. The head portion includes two projecting portions and a channel formed therebetween. A surgical blade is positioned within the shaft adjacent the head portion, with a cutting edge located within the channel. The knife may be selectively used in conjunction with a two part spatula-type retractor and an image scope. The knife and retractor are pushed into the human body with their form and dimensional relationships serving to separate adjacent vital tissues away from the facia tissue. The form and dimensions of the knife serve to guide the facia into its channel. As the knife is pushed forward, the facia is directed into the cutting blade formed at the base of the channel.

### RELATION TO OTHER APPLICATIONS

The present application claims the benefit of the filing date of U.S. Provisional Application Ser. No. 63/458,959, filed Apr. 13, 2023, which is herein incorporated by reference.

### FIELD

The present disclosure relates to the field of orthopedic surgery, trauma surgery and vascular surgery. In particular, the contemplated invention may be used for making incisions and releasing fascial tissue to relieve pressure in closed fascial compartments. Closed fascial compartments are commonly found throughout the body, including four compartments of the lower leg or three compartments of the forearm. Other closed compartments generally include three compartments of the thigh or two compartments of the arm.

### BACKGROUND

Compartment syndrome may be classified as acute or chronic depending on the cause of the increased pressure within the compartment and the duration. Acute compartment syndrome (ACS) is a medical emergency requiring immediate surgery that includes long incisions, multiple surgeries, wound vacuum assisted closure (VAC) treatment, and a higher rate of complications/infection. ACS may occur in critically sick or injured people and may be associated with a high rate of morbidity. The most common causes of ACS include fractures, soft tissue trauma, long periods of limb compression due to altered state, arterial injury, burns, anticoagulation or extravasation of intravenous fluid. Chronic exertional compartment syndrome (ECS) is a recurrent increase in compartment pressure symptoms due to exercise or increased activity. ESC often occurs in distance runners (often high school and college level athletes), other athletes and military recruits in the lower leg or in weightlifters in any compartment, although more commonly in the forearms.

Compartment syndrome is an elevation of the interstitial pressure in a closed Osseo fascial compartment that results in microvascular compromise. A person's extremities are divided into compartments by fascial tissue that can be relatively thick and noncompliant, analogous to plastic wrap around a filet mignon. Contained within each compartment are muscles, nerves, arteries, veins, and capillaries, among other tissues. The pathophysiology of compartment syndrome involves an insult to normal local tissue homeostasis that results in increased tissue pressure that may disrupt capillary blood flow and result in local tissue injury and necrosis caused by oxygen deprivation. In essence, the cells carrying oxygen to the end target tissues in need, cannot receive appropriate blood flow, potentially resulting in tissue ischemia.

In ACS, a cascade of events may result in a self-perpetuating downward spiral in a person's health, if not addressed surgically. In an example, an insult to the local tissue, such as a bone fracture, may result in bleeding and swelling of the surrounding soft tissues. Because fascial tissue is not compliant, as various other tissues expand and swell, the fascia does not expand with it. The net result is an increase in pressure within a local compartment. As pressure increases, less nutrients get to the end target tissue (muscle, nerves, etc.) potentially resulting in ischemia and necrosis. The loss of nutrients may result in more swelling, again without accommodation by the noncompliant fascia, and a further increase in pressure. The deteriorating process may be stopped with emergent, preferably complete, release of the fascial compartments. The release process may occur anywhere skeletal muscle is surrounded by substantial fascia such as the buttock, thigh, lower leg, shoulder, arm, forearm, hand, foot, or lumbar spine.

Treatment for full blown compartment syndrome is typically and emergent fasciotomy. The affected compartments may be released by cutting the fascia covering the compartment via long incisions that expose the entirety of the compartment to avoid neurovascular injury and ensure release. These surgical openings/incisions are normally left open or covered with a VAC device. The patient is typically brought back to the operating room 48-72 hours later for a second surgery including irrigation and debridement of nonviable tissue and attempted wound closure. Often two or three return trips to the operating room may be required.

CECS is a version of the ACS condition that results when an increase in activity causes an increase in compartment pressure, but never enters a self-perpetuating or downward spiral of acute compartment syndrome. In this scenario, the increase in activity, commonly seen in the lower leg of runners, cause the arteries to dilate and muscles to swell. Again, the noncompliant fascia does not accommodate the swelling, resulting in an increase in pressure within the compartment and disrupt nutrient and oxygen delivery to the adjacent tissues. The affected person may begin to develop heaviness and aching and neurologic symptoms like numbness and tingling and even weakness until the person can no longer continue the exercise. Once the person stops, the pressure reduces and the symptoms subside, until they attempt to resume the activity. Once diagnosed, the treatment for this condition is to attempt physical therapy and relative rest. This treatment may not eliminate the issues and, as activity resumes, the symptoms may return. Further treatment may typically follow two options: (1) discontinue the activity forevermore, or (2) undergo compartment releases via fasciotomy of the involved compartments. The surgery is typically performed through smaller incisions without clear visualization of the neurovascular structures and whether or not the fascia has been completely released. This is usually performed with long thoracic Metzenbaum scissors with the tips held open just slightly as the surgeon pushes the device along the fascia. The process may be difficult, since the surgeon needs to evade injury to any neurovascular structures—typically by staying in safe zones—and to avoid slipping off the fascia that may result in harm (either deep or superficial) to the fascia or the fascia not being sufficiently released (from the point where the scissor tips slipped off). Another type of knife that may be used includes a ball-like tip and an adjacent slot. However, it may be difficult for this device to stay on the fascia, and it does not afford a desired level of protection to the surrounding tissues, including neurovascular structures.

In acute traumatic compartment syndrome, if the patient has full blown compartment syndrome, or a contaminated wound that will require debridement of the tissues, long incisions and open debridement must remain the appropriate course of action.

### SUMMARY

In embodiments, one or more forms of a device may be used in cases of incipient compartment syndrome and other applications. The device may be used when a patient is beginning to develop signs of the condition, in case where a patient is in an intensive care unit (ICU) and needs to have a release performed at the bedside or in cases of ECS. In embodiments, one or more forms of the device may be used in all musculoskeletal compartments, including the lower leg, thigh, buttock, lumbar spine, shoulder, arm or forearm. One or more forms may be appropriate and/or beneficial for hand or foot compartments.

In embodiments, one or more forms of a device may be intended for use as an instrument for compartment release, while potentially reducing the chance of unintentional neurovascular injury and/or while potentially increasing the likelihood of a full or complete release of the compartments.

In one embodiment, the device may include a long handle. In another embodiment, a tab handle many be provided. The long handle embodiment may be provided in a number of lengths. In embodiments, at least two lengths are contemplated to account for variation in a patient's height (and thus the length of the compartment) and variation in the compartments to be released (e.g., the thigh vs. forearm, etc.). The knife embodiment may be used to release fascia overlying compartments through small incisions. The tab handle embodiment may be used when a fascia needs to be visualized along the course of the incision, such as in the scenario of full blown compartment syndrome or even in standard fracture work. A tab handle embodiment may be used to release exposed fascia and to also expose the deep layers there below.

One long handle knife embodiment may be provided in multiple shaft lengths. Two contemplated shaft lengths are in the range of about 25 cm and about 32 cm long. An extended tab may be provided at one end of the shaft. The tab is preferably squeezed between the thumb and index finger when pushing the device into contact with the tissue to be released. A head is provided at the opposite end of the shaft from the tab. The shaft is preferably curved at the tab (the tail or pushing end) end to allow for ease in moving the device in a direction parallel to the fascia while holding it in a plane outside of the skin and subcutaneous tissue. The geometric shape of the shaft may be formed to bending. In embodiments, the shaft may have a triangular or trapezoidal cross section shape in at least the central portion of the knife shaft or body. The shape of the shaft may provide a relatively flat bottom surface that may increase stability of the knife as it glides along muscle belly, below the fascia, or the like, and may also assist to maintain the blade end on the fascia. The provision of curved surfaces at the ends of the shaft may further enhance the movement and stability during use.

Other structures may be provided for stiffness, stability and overall operational enhancement. For example, a set of wings or similar structures may protrude laterally from the shaft relatively close to the tail end. The protruding structure(s) may provide a griping surface for a surgeon's hand to help guide the knife and to stabilize the knife during the push along the facia tissue. Typically, the wings are contemplated to be released as the shaft slides further into a provided incision and under tissue at the final portion of the overall facia release. The central portion of the shaft is preferably flat and relatively straight.

In embodiments, at the front of the knife device, the head end may include an upward curvature. The head end curvature is contemplated to direct the head up into the deep surface of the fascia. The head may be provided with a pair of projections, separated by an open channel. The head projections have a “double duck head” appearance. A portion of the head may project forward so as to press into the fascia from a position below. This forward projection of the head is intended to deflect any vital structures that reside deep to the fascia to deflect these structures below the knife and so that they glide the under the knife shaft. The leading projection is contemplated to be relatively higher than the second or trailing projection This relationship is contemplated to assist in deflecting any vital structures superficial to the fascia up and over the second projection and allowing these structures to glide along the top part of the shaft, rather than down into the slot between the two head portions.

In embodiments, a slot or channel may be provided between the two head projections. The channel may curve downwardly into the body of the knife head. A scalpel blade is provided at the base of the slot. In operation, the fascia, which is a continuous sheath, is fed into the slot towards the scalpel at the base. As the head of the knife is moved forward, the facia will continue to glide into the slot and be cut by the scalpel for release. The scalpel or knife edge may be formed as part of the head or may be a separate blade structure that is retained within the shaft and head of the device. In embodiments, a standard number 15 scalpel blade may be retained within the shaft with the cutting edge being fixed at the base of the slot.

The two head projection may have a number of shapes while serving to promote deflection of the adjacent structures and to direct the facia into the channel formed between the two projections. In embodiments, the leading projection may include a lip portion with a trailing depression. The leading lip portion is contemplated to sit relatively higher than the trailing depression and to also sit higher that the top surface of the trailing head portion. The trailing lip on the first projection is also contemplated to be relatively higher that the trailing projection portion. This dimensional relationship, along with any provided projections or curvatures, may serve to assist in deflecting encountered vital structures above or below the head of the knife while guiding only the fascia into the channel during the pushing forward of the knife. The form of the head projections and the channel preferably direct only the facia into the channel so that it may be cut or released by the scalpel blade at the base of the channel.

The tab embodiment may include the characteristics of the long handle embodiment in the front portion of the knife, such as a triangular bottom, a curve up to the head, a two projection shape and a blade recessed within the central channel. The holding tab may take any number of forms and further may be provided in the area above the head portion or slightly behind the head. During use of the tab embodiment, the knife may be pulled along the tissue for release of or to “unzip” the fascia, visualizing the fascia through a proved incision and while at least superficially visualizing and protecting structures deep to the fascia.

In embodiments, an interlocking spatula type elevator or speculum retractor may be provided to displace vital structures off the fascia and to hold the structures retracted while a knife is used to release the fascia. A retractor may be used to displace and retract the vital tissues and to both guide the knife and assist in maintaining the release within safe zones. The retractor is contemplated to be typically used with a long handle knife embodiment. The retractor may be used in situations where the surgeon is inclined to have an additional layer of protection for the facia adjacent structures. The retractor may be formed in two parts. Further, the retractor may have a number of desired lengths, so as accommodate the length of the knife.

In embodiments, a first part or portion of the retractor may have a rectangular hollow handle, that is directed perpendicular to an elongated body that ends in a curved, spoon shaped, head. The body of the first portion may form a semicircle trough that is deep enough to accommodate the head of the knife. The first retractor portion may be slid along the inferior/deep surface of the fascia. The tip of the spatula head may form a spoon shape and extend slightly beyond various surfaces of the spatula body and its head portion. The tip of the retractor head may press into the fascia for deflecting the vital structures outside of the device. A second retractor portion or part may be formed to engage with the first portion. For example, the second portion may fit inside a hollow in the handle of the first portion. The engagement preferably serves to guide the second retractor portion along the same trajectory as the first portion. The second portion may also have a semi-circular form along its length. A spoon tip on the second proton may be formed to extend slightly deeper than the rest of the length of second portion. This second portion of the retractor may be slightly shorter than the first or base portion so that the two tips do not touch each other, while both serving to elevate tissues off of the fascia.

The two handle portions of the retractor embodiment preferably lock together once completely inserted. A knife may then be inserted into the channel or cavity created by the form of the body of the two retractor parts, with the fascia visible between them. The knife may then be pushed inside the cavity for the desired length of the release. The form and dimensions of the retractor preferably serve to deflect vital structures above or below the retractor and direct the facia into the interior cavity. As the knife is inserted into the retractor the form of the knife guides the fascia into the channel formed between the two head portions. The knife is then pushed forward into the retractor for the desired length of the facia release.

### BRIEF DESCRIPTION OF THE DRAWINGS

For the purpose of illustrating the contemplated invention, there is shown in the accompanying figures a number of forms that are presently preferred; it being understood that the invention is not limited to the precise arrangements and instrumentalities shown.

FIG. 1 is an isometric view of an embodiment of a fasciotomy knife as contemplated by the present disclosure.

FIG. 2 shows an enlarge view of a head portion of the knife of FIG. 1, having a cutting blade inserted in a slot formed in the shaft of the knife.

FIG. 2A shows a cross section view of the shaft and blade as taken along line 2A-2A in FIG. 2.

FIG. 3 is a top plan view of the knife of FIGS. 1 and 2.

FIG. 4 is a partial isometric view of the head portion of the knife of FIGS. 1-3.

FIG. 4A is the isometric view of the head portion of the knife of FIG. 4 illustrating the blade being inserted into the slot formed in the shaft.

FIG. 5 show the insertion of the knife of FIGS. 1-4 into a human leg, illustrating the use during a fasciotomy procedure.

FIGS. 6A-6C show various views of a further knife embodiment as contemplated by the present disclosure.

FIGS. 7A-7C show various views of a still further knife embodiment as contemplated by the present disclosure.

FIGS. 8A-8C show side elevation views of a two part retractor embodiment for use in a fasciotomy procedure.

FIG. 8D is a partial bottom plan view of the retractor as seen from the position of line 8D-8D in FIG. 8C.

FIG. 8E is a cross section view of the retractor as taken along line 8E-8E in FIG. 8C.

FIG. 8F is a cross section view of the retractor as taken along line 8F-8F in FIG. 8C.

FIG. 8G is a partial top plan view of the retractor as seen from the position of line 8G-8G in FIG. 8C.

FIGS. 9A-9D show the insertion of the retractor of FIGS. 8A-8C and the knife of FIGS. 1-4 into a human leg, illustrating use of the retractor and a knife embodiment during a fasciotomy procedure.

FIG. 10A is a partial cross section view of the knife and retractor in use as taken along line 10A-10A in FIG. 9C.

FIG. 10B is a cross section view of the knife and retractor in use as taken along line 10B-10B in FIG. 9D.

FIG. 11A shows an isometric view of a knife embodiment along with an adjacent image producing scope.

FIG. 11B is an enlarged isometric view of the head portion of the knife of FIG. 11A, showing internal channels for receiving the wand portion of the image producing scope.

FIG. 11C is an enlarged cross section view of the knife as taken along line 11C-11C in FIG. 11B.

FIG. 11E is an partial, enlarged view of a cross section of the knife as taken along line 11E-11E in FIG. 11C.

FIG. 12A is an enlarged isometric view of the head portion of a further knife embodiment for use along with an image producing scope or similar device.

FIG. 12B is an enlarged cross section view of the knife of FIG. 12A as taken along line 12B-12B.

FIG. 12C is an partial, enlarged view of a cross section of the knife of FIG. 12A-12B as taken along line 12C-12C.

### DETAILED DESCRIPTION

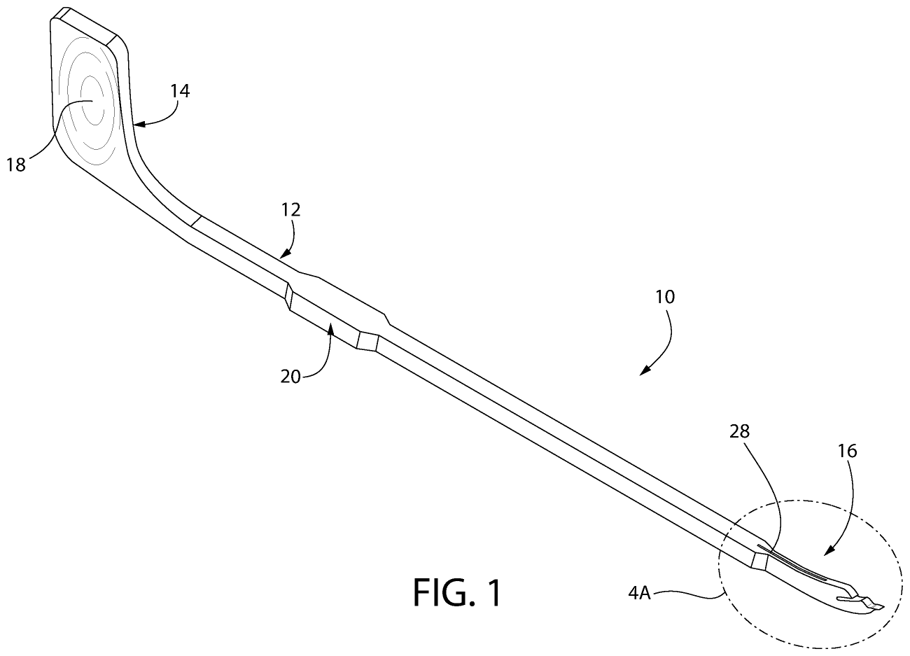

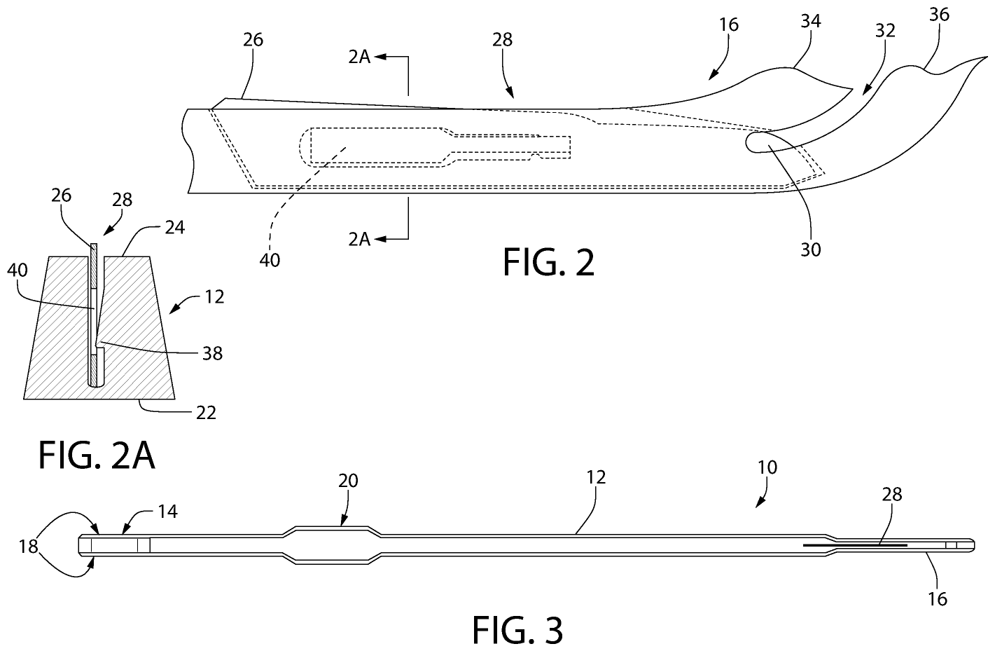

In the various accompanying figures, where like element numbers refer to similar parts of features, there is shown a number of embodiments of a fasciotomy knife, a retractor and other elements that may be utilized along with various knife embodiments. In FIGS. 1 and 3 there is shown an embodiment of the fasciotomy knife, which is generally identified by the numeral 10. The knife 10 includes an elongated shaft or handle 12 having a tab portion 14 at one end and a head portion 16 at the opposite end. The tab 14 may include contoured side surfaces 18 to facilitate gripping, for example, by a thumb and one or more fingers of a surgeon's hand. A winged or projecting stabilizer 20 is provided along the shaft 12, preferably at a position relatively closer to the rear end of the shaft or the tab 14. The head 16 may include a reduction in width as compared to the main portion of the shaft 12. As illustrated in the cross section of FIG. 2A, the shaft 12 may include a triangular or trapezoidal cross section to aid in stiffness or to otherwise provide stability during use. The shaft may—as a function of the material and shape—include a limited amount flexing during use, preferably, resulting in a limited concave curvature on the top surface. The base or lower surface 22 is shown as having a greater lateral dimension than the top surface 24. The shaft may be made of any number of materials, including surgical graded steel and biocompatible materials, such as nylon 16, polyetheretherketone (PEEK), polyvinylchloride (PVC); polyethylene (PE), polypropylene (PP), or other thermoplastics, etc. The shaft may be molded as a single piece, machined, 3D printed or otherwise formed.

FIG. 2 shows an enlarged view of the head portion 16 of the knife 10 of FIG. 1, with a cutting blade 26 shown (mostly) in phantom, positioned within a slot 28 formed in the shaft 12 adjacent the head portion 16. The cutting edge 30 of the knife blade 26 is positioned within a channel 32 formed between two projections 34, 36 of the head 16. The cutting blade 26 as illustrated is a number 15 type surgical scalpel blade. Other blade forms may be utilized, with a corresponding adjustment to the form of the slot 28 within the shaft body 12 and head 16. It may also be possible to 3D print the body of the knife with the blade formed in situ. As shown in FIG. 2, a projecting nub 38 is formed within the slot 28. The nub 38 extends into an opening in the center of the blade 26 to fix the position of the cutting edge 30 in the channel 32. The cutting edge 30 is shown as facing upwardly in the channel 32. As shown in FIGS. 2 and 2A, a tail portion of the blade 26 may project above the top surface 24 of the shaft 12.

In embodiments the two head projections 34, 36 may each be described as having a duck head form, with a bulbous base, a projecting bill, and a tapered transition therebetween. In FIG. 2, it is shown that the rearward projection 34 has a relatively gradual transition, between the base and the projecting bill. The forward projection 36 is shown as having a sharp transition between the base and an elongated projection of the bill. The bill of the forward projection 36 forms the forward edge of the knife 10.

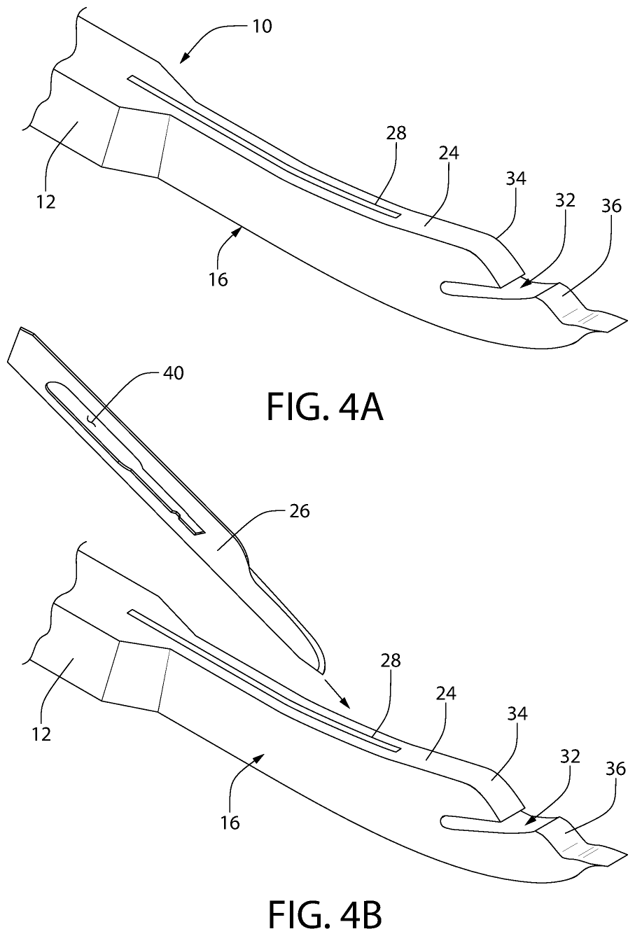

FIG. 4A shows an enlarged view of the head portion 16 of the knife 10 of FIGS. 1-3. In FIG. 4B the blade 26 is shown as being inserted into the slot 28 formed in top surface 24 of the shaft 12, adjacent the head portion 16. As shown in FIG. 2, the cutting edge 30 is facing upwards and is to be positioned within the channel 32 between the two projections 34, 36.

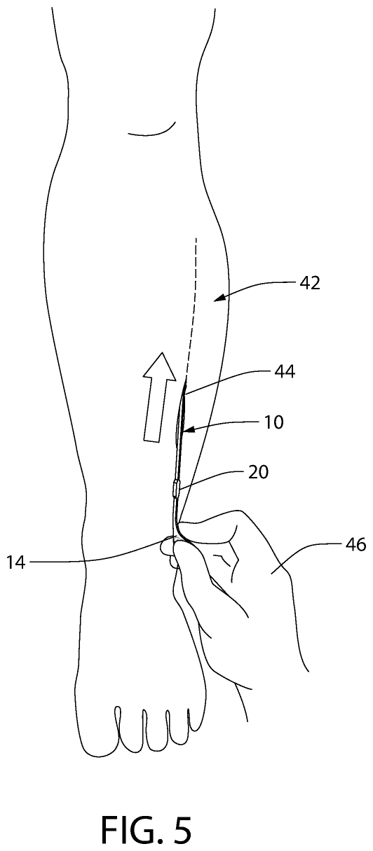

FIG. 5 shows an example of the knife 10 in use for releasing fascia tissue within a portion of the calf of a human leg 42. An initial incision creates an opening 44, preferably at the lower end of the leg 42 with sufficient depth below the skin to provide access to the fascia tissue. As shown, the head portion 16 of the knife 10 has been inserted into the opening 44 and directed towards the facia tissue. The knife is held by the surgeon 46 at the rearward tab 14. The winged stabilizer 20 assists in maintaining the knife 10 in a stabile position while inserting the knife 10 further into the opening 44. The facia is directed (not shown in this figure) between the two head projections 34, 36 and into contact with the cutting edge 30 at the base of the channel 32. The forward projection 36 is preferably positioned below the facia tissue, with the rearward projection 34 guiding the facia into the channel 32.

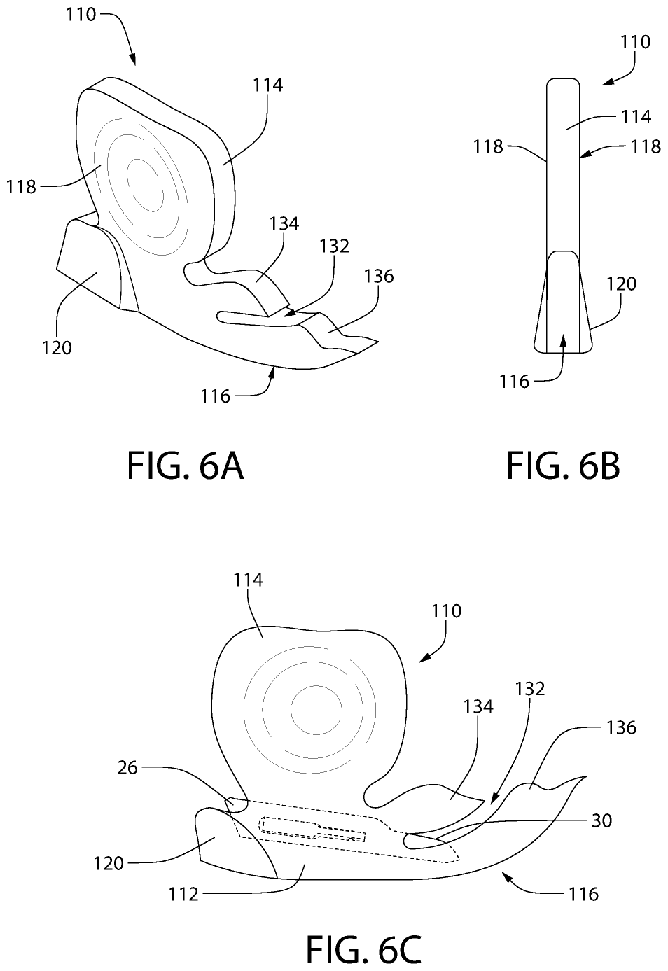

FIGS. 6A-6C show various view of an embodiment of a knife 110, having a relatively short shaft or base portion 112. Hence, the elongated shaft 12 of the knife 20 of FIG. 1 has been substantially reduced. A tab portion 114 is positioned adjacent the head portion 116. A flared base 120 is provided toward the rear of the shaft body of the knife 110. The projection of the flare 120 relative to the shaft body 112 is illustrated in the front view of FIG. 6B. FIG. 6C shows a knife blade 26 positioned within the body 112 of the knife 110, with the cutting edge 30 positioned at the base of a channel 132 formed between the two projections 134, 136 of the head portion 116. The knife blade 26 may be positioned within a slot (not designated) in the body 112, with a rearward opening or slot proved for insertion of the blade 26. Retaining structures, such as the position nub 38 of knife 10, may be provided in the blade slot. Again, the structure of the knife 110 may also be formed from any number of materials, which are preferably biocompatible. It is also possible to form the body and blade together by 3D printing or otherwise formed.

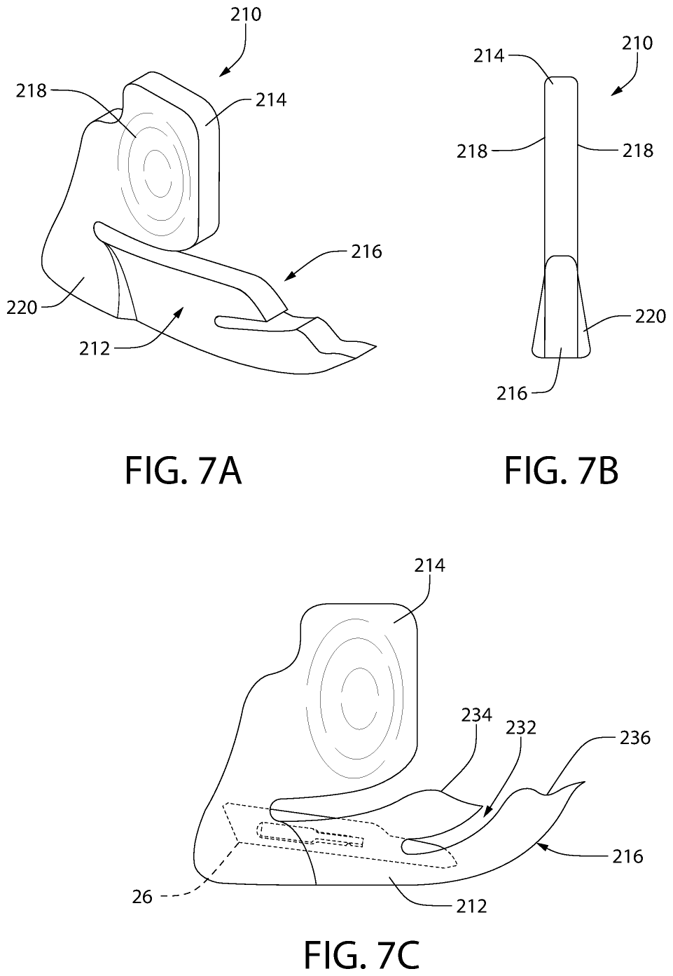

FIGS. 7A-7C show a further embodiment of a knife 210, similar to that of the knife 110, having a relatively short body 212. A tab portion 214 is positioned at the rear end of the shaft body 212, with the head portion 216 formed at the opposite end. A flared base 220 is provided at the rear of the shaft body 212, below the tab 214. The projection of the flare 220 relative to the body 212 is illustrated in the front view of FIG. 7B. FIG. 7C shows a surgical blade 26 positioned within the body 212, with the cutting edge 30 positioned at the base of the channel 232 formed between the two projections 234, 236 of the head portion 216. The blade 26 may be positioned within a slot (not designated) in the body 212, with a rearward opening or slot proved for insertion of the blade 26. Retaining structures may be provided within the blade slot or otherwise. Again, the structure of the knife 210 may be formed from any number of biocompatible materials, and may be molded, machined or formed by 3D printing.

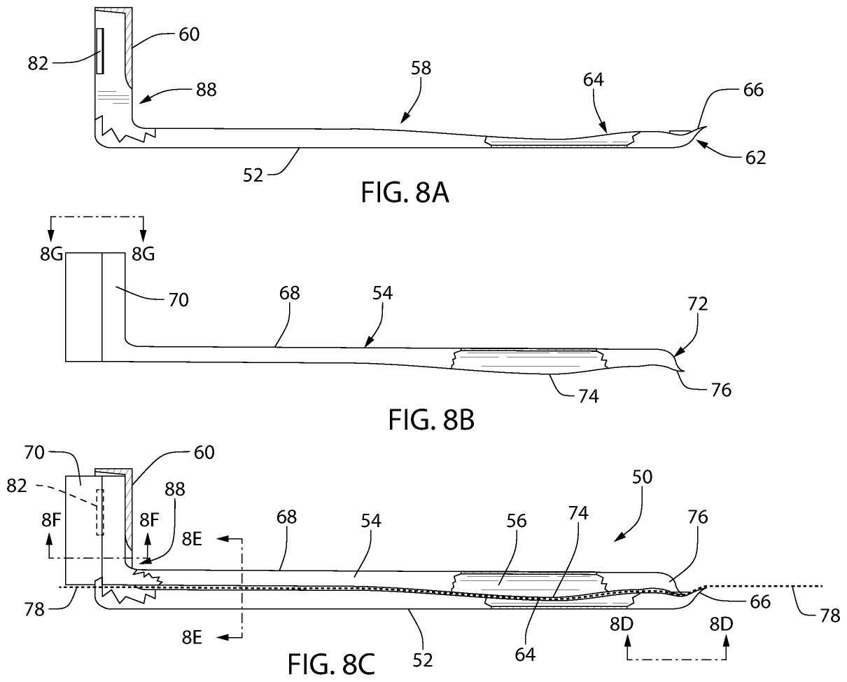

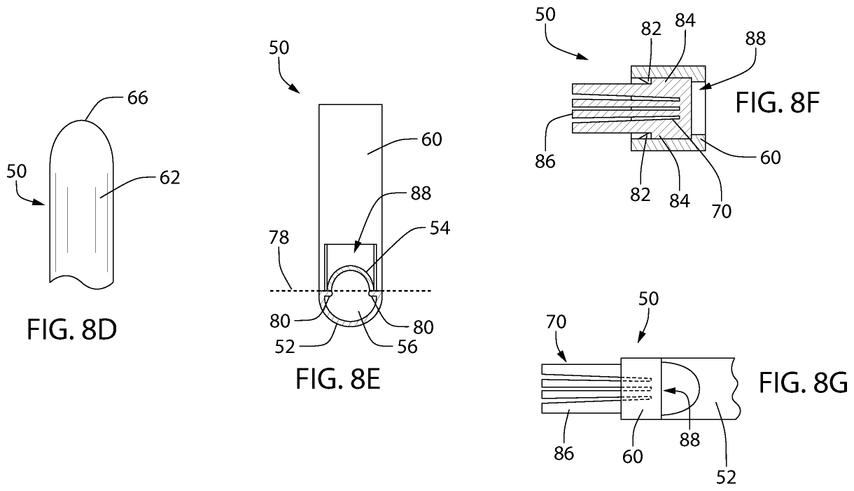

FIGS. 8A-8C shows side elevation views of the constituent parts of a spatula or speculum type retractor 50 formed from a base or bottom member 52 and a cap or top member 54. Dimensionally, the two parts 52, 54 form a hollow center channel or cavity 56 (sec, e.g., FIG. 8E). The cavity is formed to receive a knife, for example the knife embodiment 10 of FIG. 1. Depending on the dimensions of the retractor 50 and its cavity 56, the knife shaft may be formed without a stabilizer 20, as in FIG. 1. As discussed further below, the shape of the shaft and/or the shape of the head portion of the knife may create positional stability for the knife as it is moved into operational position within the retractor cavity 56.

In FIG. 8A there is shown in partial cross section of the embodiment of the base member 52 of the retractor 50. The base 52 includes an elongated, upwardly facing open trough 58 and a rearwardly positioned handle 60. Towards the forward end 62 of the base member 52 there is provided a recess 64 in the height of the side walls, resulting in a decrease in depth of the trough 58. The forward end 62 of the base 52 is shown as having a tapered, projecting lip 66. In FIG. 8B there is shown in partial cross section of the embodiment of a cap member 54 of the retractor 50. The cap 54 includes an elongated body 68 and a rearward handle 70. Towards the forward end 72 of the cap 54 there is provided an extension 74 of the sidewall, resulting in an increase in height of the sidewall. The forward end 72 of the cap is also shown as having a tapered, projecting lip 76.

In FIG. 8C, the two retractor parts 52, 54 are joined in an operational position. The dimensions of the base 52 and cap 54 preferably match so as to mate and/or align the two parts. The extension 74 of the cap 54 aligns with the recess 64 in the sidewalls of the base member 52. The lip 62 of the base member preferably projects forward of the projection of the lip 74 on the cap 54. Further, the two handle members 60, 70 engage one another to fix the relative position of the parts 52, 54. The relative position of the facia tissue is identified by the dotted line 78. As shown, the facia 78 is positioned (moves) between the two parts of the retractor 50.

FIG. 8D shows a bottom view of the retractor 50 looking at the forward end 62 of the base member and the projecting lip 66. Because the lip 66 of the base projects forward of the lip 76 of the cap 54, only the base member is visible in this partial view. In the cross section of FIG. 8E there is shown the mating of the two parts 52, 54 of the retractor 50, with the facia tissue 78 illustrated in between. The semi-circular trough 56 of the base member 52 is closed by the cap 54, with the cap wall also being shown as semi-circular. A pair of inwardly projecting tabs 80 are formed on the top edge of the sidewalls of the base member 52. As shown in the embodiment, the tabs 80 support the position of the cap 54. The facia 78 is shown suspended across the formed cavity 56.

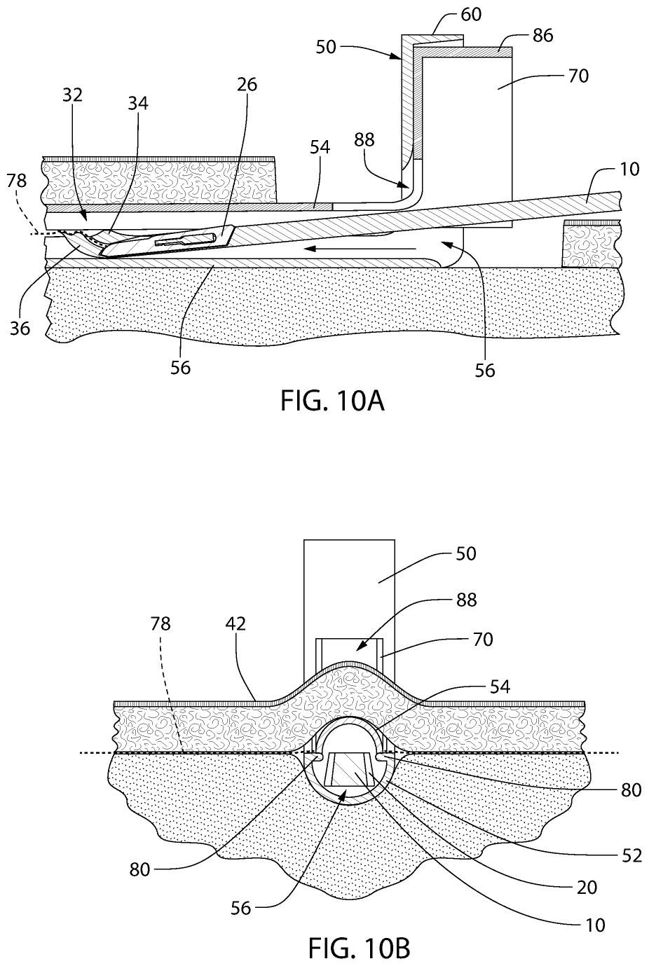

In the cross sections of FIG. 8F, there is shown the engagement of the handle 60 of the base 52 with the handle portion 70 of the cap 54. Generally, the dimensions of cap handle 70 fit inside the contours of the base handle 60. A pair of prominences or tabs 82 are provided on the inside wall of the base member 52. The cap handle 60 includes a similar prominences or shoulders 84 that engages with the tabs 82. The sets of prominences or engagement surfaces may be rounded or otherwise formed to assist in engagement as the one handle portion 54 is moved to the fixed position relative to the base portion 52. (Such as that shown in FIG. 8C. See also FIG. 10A.) A series of fenestrations or slotted openings 86 are formed on the projected end of the cap handle 70 to permit flex for ease of insertion and separation of the two parts at the engagement of the tabs 80 and shoulder 84. The inside walls of the handle 60 of the base member for a stop to fix the position of the handle 70 of the cap portion 54. Preferably, the stop or ridge may be angled relative to vertical so that the shaft of the cap portion 54 is pushed down into the fascia, assisting in scraping the neurovascular structures off the fascia.

As can also be seen in FIGS. 8A and 8G, an opening 88 is provided on the forward face of the handle 60 of the base 52 for insertion of the cap 54 through the base handle 60 and onto the top edge of the tabs 80 on the sidewalls of the base member 52. The opening 88 is shown (such as in FIG. 8C) as having a curved forward end. it is an edge of an opening in the top part which allows two things. One extra room to insert the knife, This opening may provide viewing access to the fascia once it is isolated between the two halves 52, 54 of the retractor 50. Further, the opening 88 may provide viewing of the knife 10 on the fascia before pushing the knife further into the retractor and completing release of the facia.

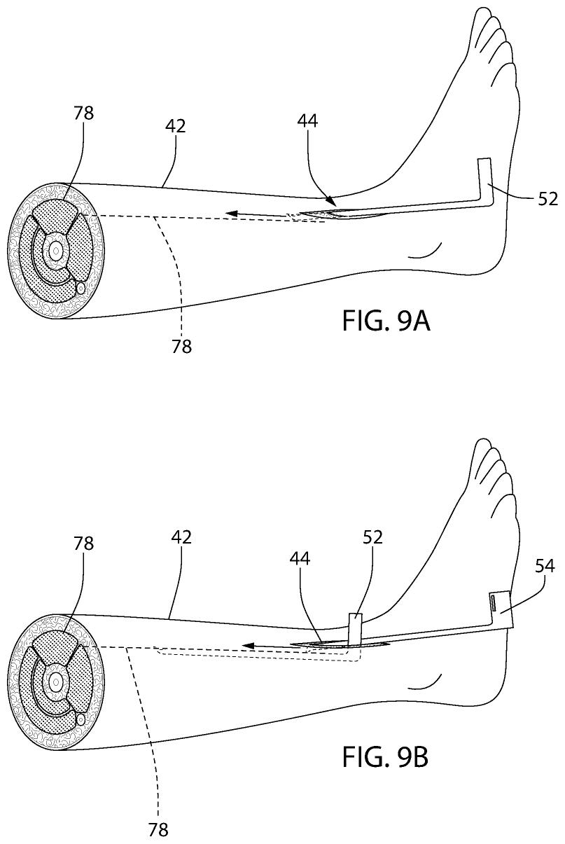

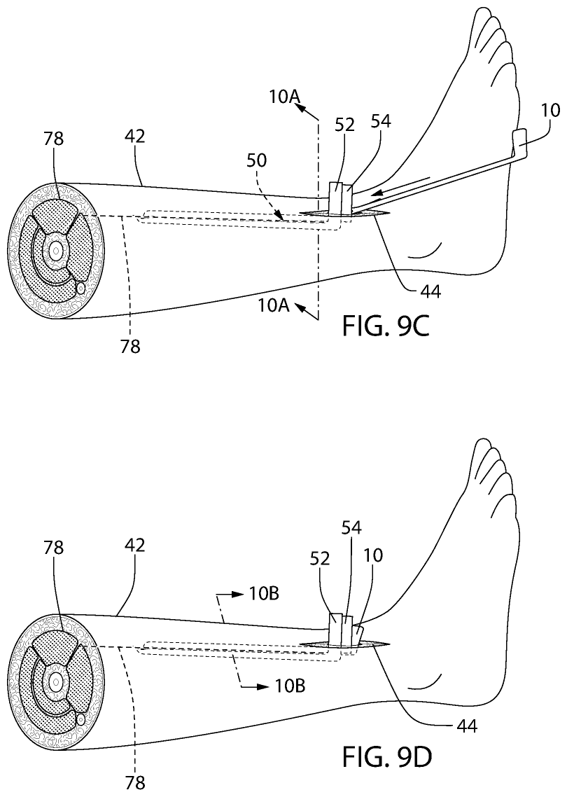

FIGS. 9A to 9D show the retractor 50 and a knife embodiment, such as knife 10, being inserted into a surgically created opening in a human leg 42. The insertion of the retractor 50 is directed from the ankle end of the shin, along the calf and toward the knee (not shown). An illustrative cross section of the leg bones and internal tissues and compartments are represented in each figure, with the facia tissue 78 designated. In FIG. 9A, the bottom or base member 52 of the retractor 50 is first inserted into an incision or opening 44 to a position below the facia tissue 78. In FIG. 9B, the upper or cap portion 54 of the retractor is first inserted into the opening 88 in the rear handle 60 (see FIGS. 8A, 8C and 8E) and moved into the incision 44 to a position above the facia tissue 78. FIGS. 8C and 8E illustrate the preferred internal alignment of the two parts 52, 54 of the retractor 50. In FIG. 9C, the knife 10 is inserted into the rear of the cavity 56 (see FIG. 8E) of the retractor 50. FIG. 9D shows the knife 10 fully inserted into the retractor, within the leg 42.

The form and dimensions of the retractor parts 52, 54 serve to deflect vital structures above or below the retractor 50 and direct the facia 78 into the interior cavity 56. As the knife 10 is inserted into the retractor 50 the fascia is guided into the channel 32 formed between the two head projections 34, 36. The knife 10 is then pushed forward into the retractor 50. As the facia is directed into the channel 32 of the knife 10, the cutting blade 26 severs the facia. The length of insertion of the knife creates the desired length of facia release.

The cross sections of FIGS. 10A and 10B show the knife 10 inserted into the retractor 50 that has previously been positioned within leg 42 (or the like). FIG. 10A shows the knife 10 within the cavity 56 of the retractor 50. The forward projection 36 of the knife head (16) moves underneath the facia tissue 78 and guides it into the channel 32 formed between the forward projection 36 and rear projection 34. As the facia tissue 78 moves into the channel 32 it is brought in contact with its cutting edge (30) of the blade 26 and is severed. The retractor 50 serves to position the facia 78 within the channel 56 and provide access to the facia tissue 78. FIG. 10B illustrates in a transverse cross section the position of the shaft (12) of the knife 10 within the cavity 56 of the retractor 50. The position of the cross section is post-cut of the facia tissue 78. The facia tissue 78 is shown with the two sides supported by the tabs 80 formed on the sidewalls of the base member 52 and on top by the sidewalls of the retractor cap 54. However, once release is accomplished by the cut of the knife 10, the facia 78 may separate and move away from the retractor 50. Preferably, the knife 10 does not completely fill the cavity 86 created by the retractor 50 in either height or width. There is preferably a few millimeters of room for lateral and vertical movement within the cavity 56. If the stabilizer or wings 20 are provided on the shaft 12 of the knife 10, it is contemplated that the lateral width of the cavity will be almost filled by these structures.

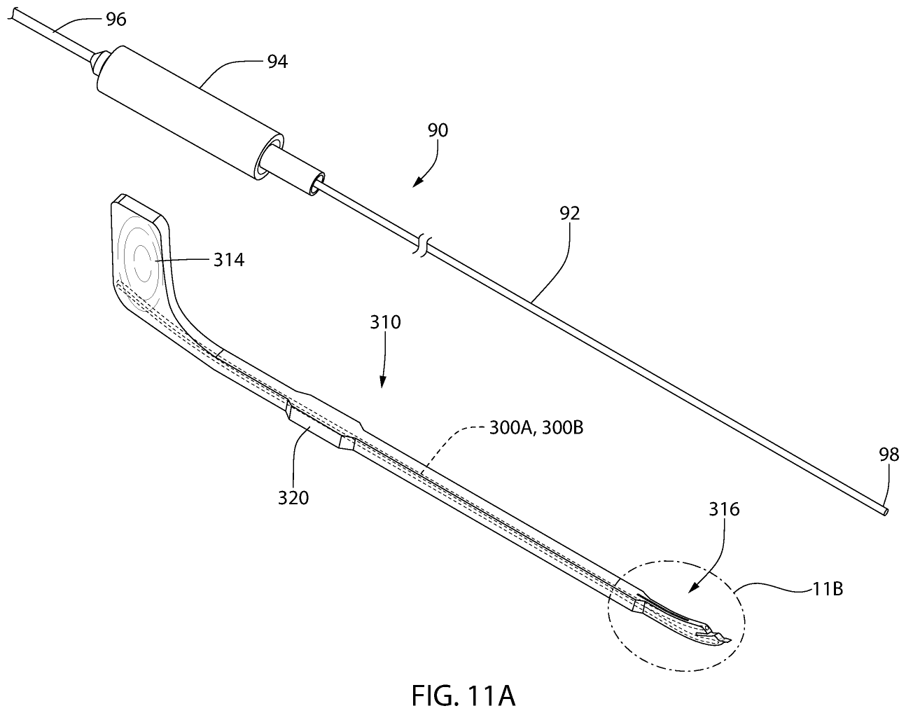

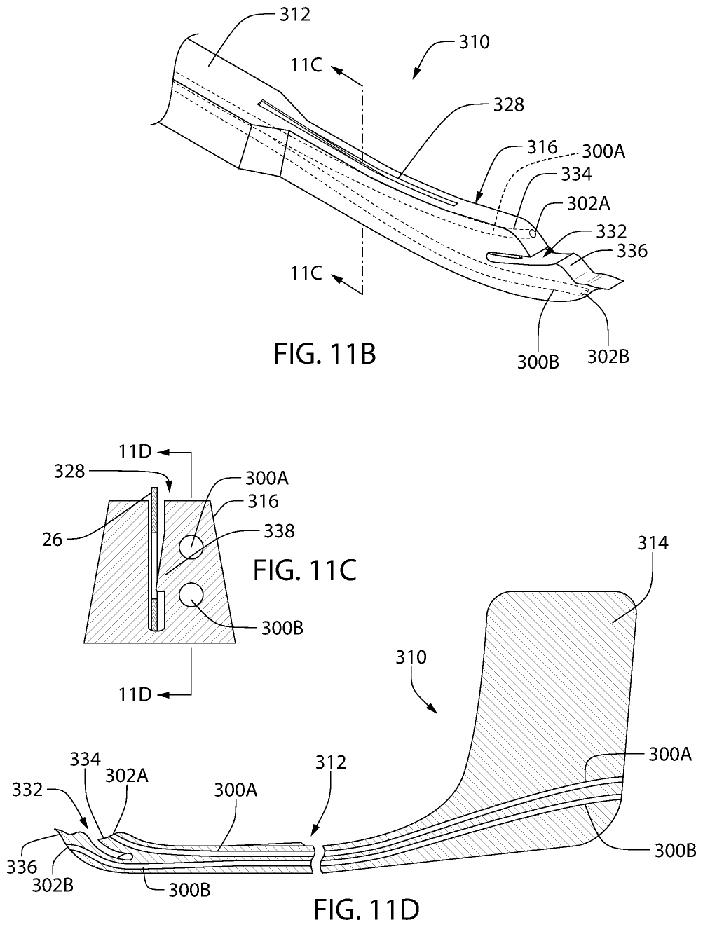

FIG. 11A shows an embodiment of a knife 310 that is formed to receive a wand portion 92 of a visual scope 90. The scope 90 may take any number of known forms, including devices commonly referred to as a nano-scope (Arthrex, Inc.) or needle scope. The wand portion 92 projects from a controller handle 94, which in turn is generally connected 96 to a power source (not shown). The forward end of the wand 92 is provided with structures 98 that provide visual images to a monitor or the like (not shown) either through the connection 96 or over a wireless communications network. The image producing structures 98, may include chips filaments or the like, and provide camera-like images on a viewing monitor. The structures 98 may vary depending on the type of scope being used. The wand 92 has a relatively small outer dimensions and is preferably flexible.

The knife 310 is illustrated to be generally similar in form to the knife embodiment of FIGS. 1-3. The knife 310 includes a central shaft 312, a rear directing tab 314 and a forward head portion 316. A winged-type or other stabilizer structure 320 may be provided along the shaft 312. The cross sectional dimensions of the shaft 312 and the head portion 316 may be formed as a trapezoid, sec, e.g., FIG. 11C, for further stabilization and/or resistance to flex. As shown in FIG. 11B, the head portion 316 may include a double projection form 334, 336, with a channel 332 formed therebetween.

As shown in FIGS. 11B and 11C, a surgical blade 26 may be inserted into a slot 328 formed at the base of the head portion 316. A retaining nub 338 or similar functioning structures may be formed in the slot 328 to fix the blade 26. As illustrated in FIG. 11D, the cutting edge of the blade is preferably positioned at the base of the channel 332, similar to the positioning shown in FIG. 2. The knife 310 is provided with two interior canals 300A, 300B that extend from the rear of the shaft 316 to the head portion 316. The terminal end 302A of canal 300A is positioned in a forward surface of the rear projection 334 of the head portion 316. The terminal end 302B of canal 300B is positioned in a forward surface of the front projection 346 of the head portion 316. Additional channels of other channel end positions may be utilized as desired.

The wand 92 of the scope 90 is inserted into one of the canals 300A, 300B, with the image tip 98 positioned at the respective canal ends 302A, 302B. The image tip 98 is positioned to provide a view of the tissues forward of the knife 310 as it moves during a fasciotomy procedure. It is possible that multiple scopes may be provided, with one positioned in each channel. The live images of the approaching tissues, as the knife is moved forward, may identify nerve or other tissues that may need realignment of the knife head 316 or a realignment of the accompanying retractor (such as retractor 50) in an attempt to avoid unintended or undesired harm or indicate that other manipulation is desired to properly position the knife.

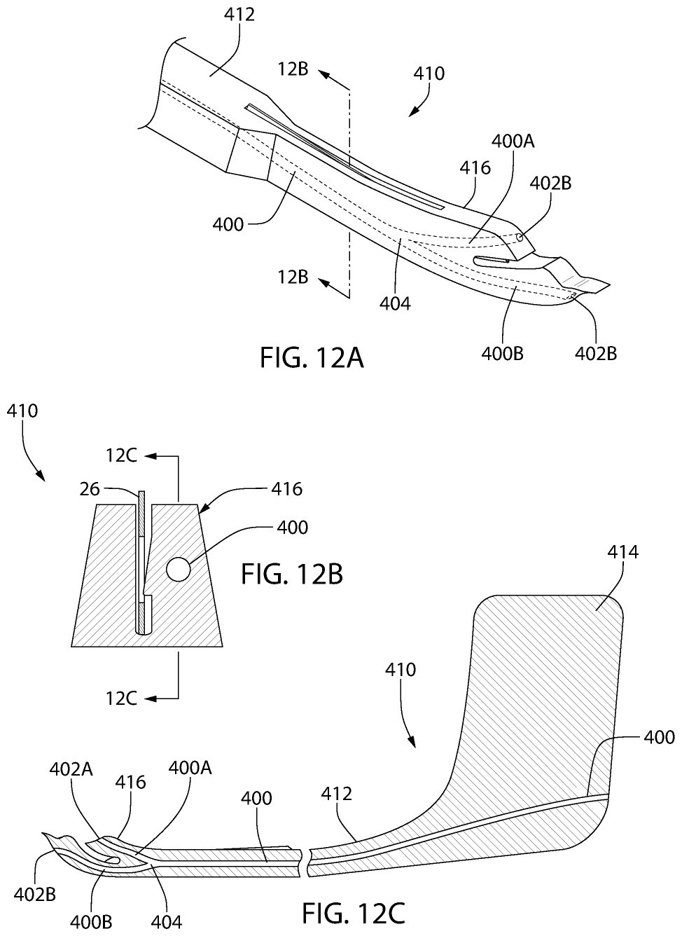

A further embodiment of the knife 410 is shown in the various views of FIGS. 12A-12C. As more particularly shown in FIGS. 12B and 12C, a single canal 400 is provided along the shaft 412 and extends to the base of the head portion 416. Adjacent the based on the head portion 41, a split 404 in the canal 400 is provided, to form two forward canal portions 400A, 400B. The canal portions 400A, 400B terminate at ends 402A, 402B on the respective head projections 434, 436. Thus, the canal portions 400A, 400B are positioned on opposite sides of the channel 432. The wand portion of the scope, such as scope 90 in FIGS. 11A-11D, may be directed into the single canal 400 and moved towards the head portion 416 of the knife 410. At the split 404 the image tip may be manipulated to move into the desired canal portions 400A, 400B, such that the tip 98 ends up in the desired position on projection 434 or 436. Alternatively, two image chips may be provided, with one directed into canal portion 400A and the other into canal portion 400B.

It is contemplated that additional scope positioning mechanisms are possible and useful. For example, more than two canals may be provided within the knife structures, with the canal ends located at another position on the knife. Although not shown in the figures, an external mount may be provided on the knife to hold the wand tip 98, to position the image tip 98 at various locations, such as, e.g., adjacent to the channel 332, 432. A canal structure or mounting structure may also be incorporated into either port of the retractor. The tip 98 of the wand 90 may also be inserted adjacent the knife (such as knife 10) within the cavity 56 of the retractor 50 (or the like).

In the figures and specification, there has been set forth a number of embodiments. These embodiments are not considered to be limiting on the potential scope of the contemplated invention. Further, although specific terms are employed herein, these terms are intended to be used in a generic and descriptive sense only and not, unless specifically stated, for purposes of limitation. Moreover, the scope of the invention is set forth in the following claims.

What is claimed is:

1. A fasciotomy knife comprising:
an elongated shaft,
a holding tab portion at one end of the shaft, and
a knife head portion at a second end of the shaft, opposite of the tab portion,
wherein the head portion comprises two projecting head portions and a channel formed therebetween, and a surgical blade having a cutting edge positioned within the channel.

2. The fasciotomy knife as in claim 1, wherein the tab portion comprises a contoured surface to facilitate gripping.

3. The fasciotomy knife as in claim 1, further comprising a winged stabilizer projecting transverse to a longitudinal direction of the shaft, and wherein the stabilizer is provided along the shaft at a position relatively closer to the holding tab than to the head portion.

4. The fasciotomy knife as in claim 1, wherein the shaft includes a trapezoidal cross section.

5. The fasciotomy knife as in claim 1, wherein the shaft, tab and head portion are made of a surgical grade steel.

6. The fasciotomy knife as in claim 1, wherein the shaft, tab and head portion are made of a biocompatible material.

7. The fasciotomy knife as in claim 6, wherein the biocompatible material is taken from the group consisting of nylon 16, polyetheretherketone (PEEK), polyvinylchloride (PVC); polyethylene (PE) and polypropylene (PP).

8. The fasciotomy knife as in claim 1 wherein the surgical blade is a number 15 scalpel blade and is retained within a slot formed in the shaft.

9. The fasciotomy knife as in claim 1 further comprising a first canal provided within the shaft, the first canal having a inlet adjacent the tab end of the shaft and a first opening adjacent the head end, the first canal formed to receive a image scope and to position the scope end within the opening at the head end.

10. The fasciotomy knife as in claim 9 further comprising a second canal within the shaft.

11. The fasciotomy knife as in claim 10, wherein the second canal is formed substantially parallel to the first canal, and wherein the first opening is positioned on a first projection of the two projection head and wherein the second opening is positioned on a second projection of the two projection head.

12. The fasciotomy knife as in claim 9, wherein the first canal comprises a first canal end portion and a second canal end portion, the first canal end portion extending from the first canal to the first opening and the second canal end portion extending from the first canal to a second opening.

13. The fasciotomy knife as in claim 12, wherein the first opening is positioned on a first projection of the two projection head and wherein the second opening is positioned on a second projection of the two projection head.

14. A fasciotomy knife comprising:
a shaft having a body portion,
a gripping tab portion projecting orthogonally from the body portion, and
a knife head portion aligned with the body portion and projecting from the body portion at an end of the shaft opposite of the gripping tab portion,
wherein the head portion comprises two projecting head portions and a channel formed therebetween, and a surgical blade having a cutting edge positioned within a base of the channel.

15. A fasciotomy knife as in claim 14, wherein the gripping tab portion comprises a lateral contoured surface to facilitate gripping.

16. A fasciotomy knife as in claim 14, wherein the body portion includes a trapezoidal cross section.

17. A device for use in fasciotomy release, the device comprising:
a spatula type retractor comprising
a top portion and a bottom portion, each portion having a handle formed at one end and a curved lip formed at their opposite end,
the top and bottom portions having opposed hemi-circular cross sections and combining for form a substantially circular channel therebetween,
the bottom portion being dimensionally longer than the top portion, such that in assembly the lip of the bottom portion projects outwardly of the lip of the top portion, and
a channel is defined between the top and bottom portions when assembled.

18. The device as in claim 17, further comprising
a fasciotomy knife to be operatively positioned within the channel formed between the assembled top and bottom portions of the retractor, the knife comprising
an elongated shaft,
a holding tab portion positioned adjacent one end of the shaft, and
a knife head portion positioned at a second end of the shaft, opposite of the tab portion, the head having two projecting head portions and a channel formed therebetween, and
a surgical blade having a cutting edge positioned within the channel.

19. The device as in claim 18, further comprising:
an image scope having a flexible wand portion and an image tip formed on the end of the image scope, wherein the knife is adapted to receive the wand portion of the image scope.

20. The device as in claim 19, wherein the fasciotomy knife further comprises
at least one canal provided within the shaft. the at least one canal having an inlet within the shaft and at least one opening adjacent the second end, and
wherein the at least one canal is formed to receive the wand of the image scope and to position the image tip within the at least one opening at the second end of the at least one canal.
